(19) United States
(12) Patent Application Publication
(10) Pub. No.: US 2026/0215831 A1
(43) Pub. Date: Jul. 30, 2026

(54) GUIDEWIRE AND METHOD FOR PERFORMING SI JOINT FUSION

(71) Applicant: ORTHOFUNDAMENTALS, LLC, Waltham, MA (US)

(72) Inventors: Jarred Sakakeeny, Hopkinton, MA (US); Matthew Paimer, Weston, MA (US)

(21) Appl. No.: 19/147,494

(22) PCT Filed: Jan. 12, 2024
(86) PCT No.: PCT/US2024/011403

§ 371 Date: Jul. 11, 2025

Related U.S. Application Data
(60) Provisional application No. 63/479,627, filed on Jan. 12, 2023.

(51) Int. Cl. A61B 17/88; A61B 17/00; A61B 17/34; A61B 17/56; A61B 17/86

(52) U.S. Cl. CPC A61B 17/8897; A61B 17/3472; A61B 17/86; A61B 2017/00867; A61B 2017/564

(57) ABSTRACT

Guidewires for facilitating the placement of implants and instruments during orthopedic surgery are disclosed. The guidewires are constructed and arranged to minimize and prevent undesired migration and/or rotation of the guidewire at a surgical site. Associated methods of performing surgery, e.g. a Sacroiliac (SI) joint fusion procedure, using the guidewire are also disclosed.

### CROSS-REFERENCE TO RELATED APPLICATION

This application claims priority under 35 U.S.C. § 119 (e) to U.S. Provisional Patent Application Ser. No. 63/479,627, titled “NOVEL GUIDEWIRE AND METHOD FOR PERFORMING SI JOINT FUSION” and filed on Jan. 12, 2023, the entire disclosure of which is hereby incorporated herein by reference in its entirety for all purposes.

### FIELD OF INVENTION

The present invention relates to wires for guiding the placement of orthopedic implants and instruments during orthopedic surgery. The invention finds particular utility in the ability to minimize and prevent undesired migration of the wire during the surgical procedure. While the invention has application throughout the body, its utility will be illustrated in the context of fusion between the ilium and the sacrum (SI Joint Fusion).

### BACKGROUND

In orthopedic procedures, it is common to prepare the surgical site by first placing a guidewire, pin, or K-wire into the surgical site under fluoroscopic guidance to help safely guide the subsequent tools (i.e. awl, drill, tap, screwdriver, etc.) and implants (screws, nails, etc) into the same targeted surgical site. The guidewire is typically placed under fluoroscopic guidance to confirm its safe passage into the intended surgical site and a cannulated tool such as a bone marrow aspirate needle or Jamshidi needle may be used to assist in placement. Guidewires, though intended to improve safety in fluoroscopically guided procedures, can cause unintended harm to the patient when the guidewire migrates after placement. Migration can sometimes occur when subsequent instruments such as a drill or tap are introduced over the guidewire causing the wire to migrate and perforate adjacent tissue or bone in which it is housed and force the wire into the delicate surrounding structures.

Alternatively, while mainly contained within the softer cancellous bone of a vertebra, long bone, or sacrum, the guidewire can be inadvertently removed during removal of the instrument which has passed over it causing the surgery to be delayed and requiring additional exposure to radiation while the guidewire is replaced.

Sacroiliac (SI) joint fusion is one such procedure in which a guidewire can be used where inadvertent iatrogenic harm can occur. Thus, there exists a clinical need for wires that resist migration. The novel methods and devices described herein prevent harm to surrounding tissues due to the migration of guidewires during sacroiliac joint fusion.

### SUMMARY

The present invention provides novel surgical tools and methods for implanting cannulated implants to transfix two or more bones. The present invention relates to methods of introducing fusion implants into a targeted surgical site through a novel expanding guidewire. The inventive guidewire has a distal end portion which can be deformed to an expanded shape with three or more expanding flanges or tines which, when expanded, provide a larger cross-sectional area relative to its proximal non-deformed portion. The projected area provided by the flanges is increased by the superelastic properties of the material that the guidewire is manufactured from, as well as increased by advancement of the wire into the surrounding bony tissue as it encounters resistance from the anatomy into which it is being advanced. Thus, if the wire attempts to migrate forward, the flanges or tines flex more open and thus resist migration of the wire. The guidewire may be constructed and arranged to anchor it at a surgical site.

The present invention also involves a method of performing surgery utilizing the inventive guidewire. The method involves the use of an introduction sheath which acts to keep the distal portion of the guidewire in its non-deformed state and facilitate the introduction of the guidewire into a cannula or jamshidi needle. As the inventive guidewire exits the confines of the jamshidi needle and enters the body, the superelastic material properties of the device and any forward advancement causes splaying of the flanges or tines. The inventive guidewire is provided to the facility/surgeon as a sterile device and pre-packaged fully contained within the introduction sheath.

The disclosure contemplates all combinations of any one or more of the foregoing aspects and/or embodiments, as well as combinations with any one or more of the embodiments set forth in the detailed description and any examples.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic drawing showing traditional orthopedic guidewires.

FIG. 2 is a schematic drawing showing a novel guidewire formed in accordance with the present invention.

FIGS. 3A, 3B and 3C are schematics showing the tip of a novel guidewire formed in accordance with the present invention.

FIG. 4 is a schematic showing the mechanical properties of Nitinol.

FIGS. 5 and 6 are schematics showing the different crystal structures of Nitinol.

FIG. 7 is a schematic showing the effect of A_{f} temperature on the loading and unloading characteristics of Nitinol.

FIGS. 8A and 8B are schematic drawings showing sheaths formed in accordance with the present invention.

FIG. 9 is a schematic drawing showing the novel k-wire constrained within the sheath formed in accordance with the present invention.

FIG. 10 is a schematic drawing showing a jamshidi needle formed in accordance with the present invention.

FIG. 11 is a schematic drawing showing the novel k-wire and sheath introduced into the jamshidi needle.

FIG. 12 is a schematic drawing showing the novel k-wire exiting the tip of the jamshidi needle.

FIG. 13 is a schematic drawing showing the use of the novel k-wire in a sacroiliac joint fusion.

FIG. 14 is a schematic drawing for a kit that can be used for performing SI Joint Fusion and including a novel guidewire.

FIGS. 15A and 15B are schematic drawings showing how surgical instruments can be used with the guidewire.

FIG. 16 is a schematic showing the steps of implanting a screw over the novel guidewire where in the guidewire is not removed during screw implantation.

FIG. 17 is a schematic showing the steps of implanting a screw over the novel guidewire where in the guidewire is removed during screw implantation.

### DETAILED DESCRIPTION

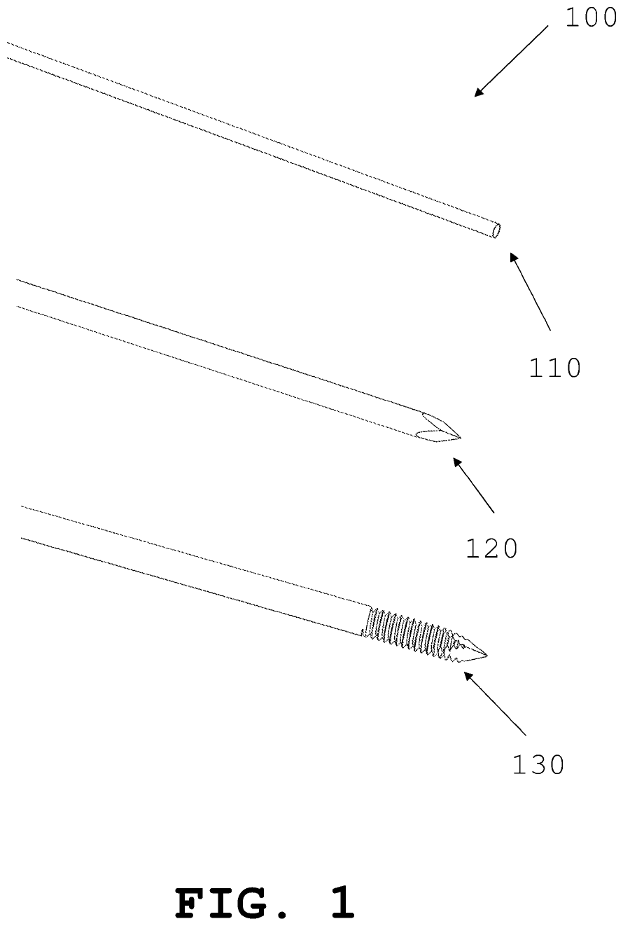

Looking first at FIG. 1 there are shown common orthopedic guidewires 100, used in orthopedic procedures. In orthopedics, it is common to use a guidewire to assist with the alignment of tools and the placement of implants. Traditionally, guidewires may have a blunt end, 110, a trocar shaped end, 120, or a threaded end, 130. While these tip geometries assist with placing the guidewire in its desired location, they do not provide any positional resistance. It is common for a trocar (or other tip shaped guidewire) to unintentionally spin when an instrument (i.e., a drill bit or a screwdriver) is placed over it and rotated. Even worse, the guidewire may advance inadvertently as an instrument (i.e., a drill bit or a screwdriver) is advanced over the guidewire. Additionally, traditional guidewires may inadvertently come out of the body when an instrument (i.e., drill bit or screwdriver) is removed from the body while over a guidewire.

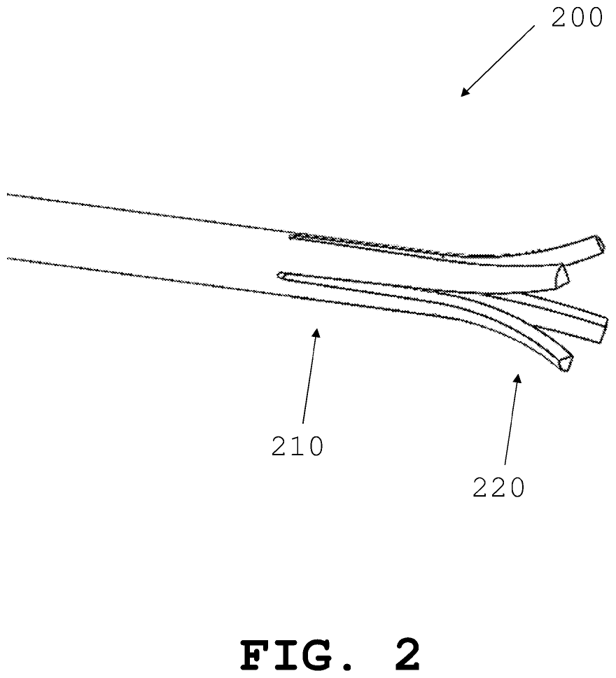

Looking now at FIG. 2 there is shown a novel guidewire 200 formed in accordance with the present invention that addresses the deficiencies of traditional guidewires. Novel guidewire 200 resists pullout and advancement, as well as provides rotational stability. In some embodiments, novel guidewire 200 is formed from a shape memory and superelastic alloy such as a nickel titanium alloy (Nitinol). Novel guidewire 200 has a distal end 210. Distal end 210 is cut to create a plurality of tines, 220. In some embodiments, guidewire 200 has two or more tines. In at least some embodiments, guidewire 200 has three or more tines, e.g. 3, 4, 5, 6, 7, 8, 9, 10, 11, 12 or more tines. In certain non-limiting embodiments, the guidewire may have an even number of tines. Increasing the number of tines increases the number of potential contact points between the tines and the bone. The more contact points, the more resistance to migration and rotation. Tines 220 may generally have a triangular or wedge shape cross-section. Tines 220 are cut into the Nitinol guidewire and then distal end 210 may be shape set to create the flared tines. The purpose of the tines is to increase the projected cross-sectional area of the guidewire. Guidewires vary in diameter, with many common orthopedic guidewires ranging from 1.0 mm to 3.2 mm in diameter. As an example, a 2.4 mm guidewire has a cross-sectional area of ~4.5 mm². That same diameter guidewire formed with 4 tines has a projected cross-sectional area of ~23.0 mm², or ~5× more cross-sectional area. That increase in cross-sectional area increases the force required to advance the wire from less than about one pound to greater than about five pounds in low quality cancellous bone. Additionally, were a traditional guidewire to advance through the soft cancellous bone, it may still push through the denser cortical bone. The same diameter guidewire formed with 4 tines will further resist advancement through cortical bone—the tines will further splay open against the inner cortical surface. Additionally, the plurality of tines creates more friction between the guidewire and the bone, thus increasing the guidewire's resistance to rotation.

In accordance with one or more embodiments, the guidewire may be capable of resisting advancement when loaded in excess of 1 lb. The guidewire may be capable of resisting advancement when loaded in excess of 5 lbs. The guidewire may be capable of resisting advancement when loaded in excess of 10 lbs.

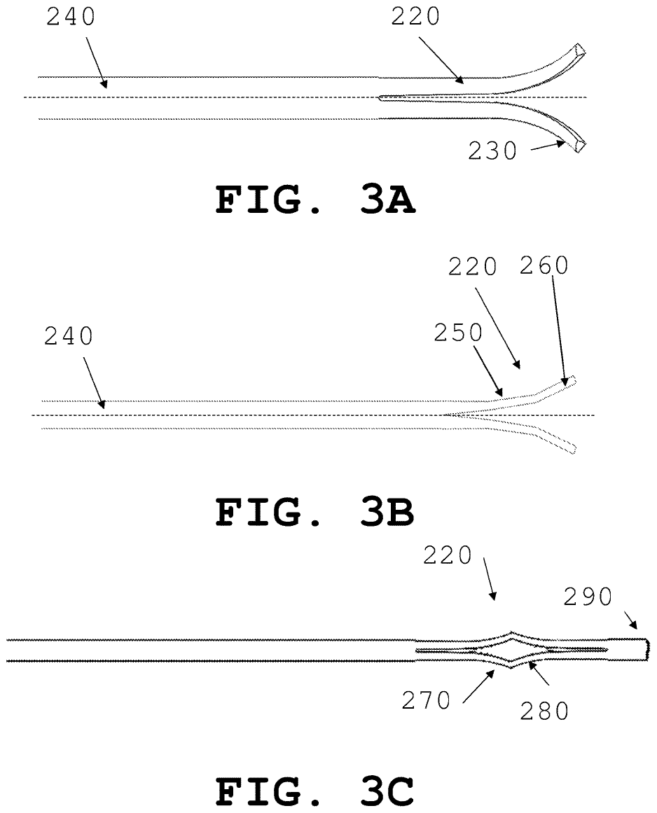

Looking now at FIG. 3A, tine 220 is shown. Tine 220 is shape set such that the rate that the tine displaces from the central axis 240 of the guidewire increases as one nears the end of the guidewire. Tine 220 may have a cross-sectional shape that is substantially triangular or wedge-shaped. The tine is not simply linearly displaced from the central axis. This creates ends of the tines 230 that are approaching perpendicular the central axis 240. Thus, if an axial force is applied to the guidewire the ends of the tines 230 will have maximum surface area in contact with the bone to resist translation and rotation. Alternatively, looking at FIG. 3B, tines 220 may be shape set with two or more linear regions 250 and 260 of different slopes of displacement from the central axis 240. For example, tines 220 may have two, three, four, five or more linear regions of varying displacement slopes. Linear region 260 is more perpendicular to the central axis 240 of the guidewire. Thus, if an axial force is applied to the guidewire the ends of the tines 260 will have maximum surface area in contact with the bone to resist translation and rotation. Alternatively, looking at FIG. 3C, the guidewire may include an expansion zone or segment in which tines 220 may have an outward flared region 270, and inward flared region 280. In these embodiments, the guidewire may have a closed fully formed tip 290. A guidewire may have a proximal portion and a distal tip, with an expansion zone or expansion segment located along a length of the guidewire. In some embodiments, the expansion segment may be located at the distal tip. In other embodiments, the expansion segment may be located up the length of the guidewire proximate to the distal tip.

In accordance with one or more embodiments, the guidewire may be dimensioned to meet intended use applications. For example, the guidewire may be of a specified diameter. In some embodiments, the guidewire may be of a circular cross-section. In at least some non-limiting embodiments, the guidewire may have a noncircular cross-section to provide additional resistance to rotation in virgin bone.

Guidewire 200 may be manufactured to a calibrated length—that way it can be used with other instruments to aid in implant length selection as will be discussed later in the application. In one specific non-limiting example the guidewire is 300 mm in length. Tines on distal end 210 may be greater 5 mm in length and preferably between 10 mm and 20 mm in length. The longer the distal end 210 is, the larger the projected cross-sectional area of the tines can be shape set.

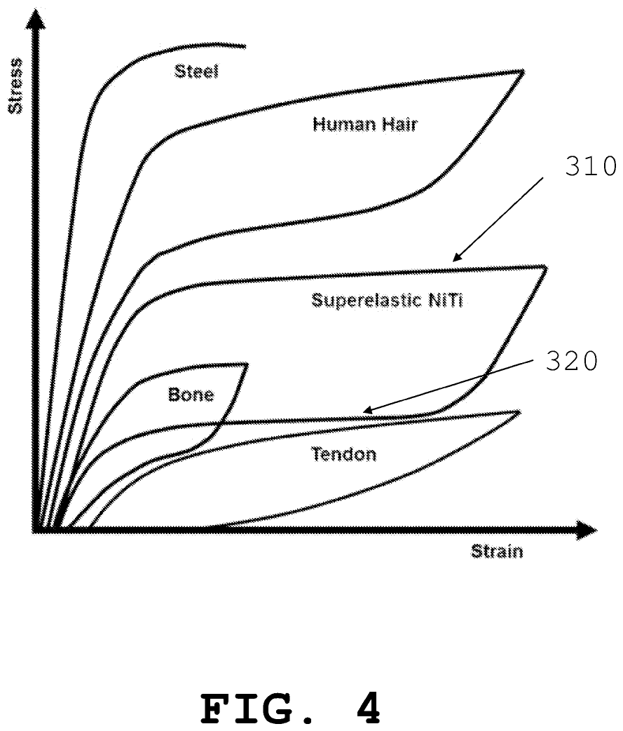

The guidewire may be made of various biocompatible materials having the requisite material properties. In some embodiments, the guidewire is made of a superelastic material. In preferred non-limiting embodiments, novel guidewire 200 is formed from Nitinol. Looking at FIG. 4, Nitinol, unlike steel and other biocompatible materials, can recover large amounts of strain. Nitinol can recover up to about 8% strain, while steel can only recover approximately 1%. When Nitinol is strained, it deforms a stress defined by the upper plateau 310. When the stress is released, it returns to its original shape with a stress defined by the lower plateau 320.

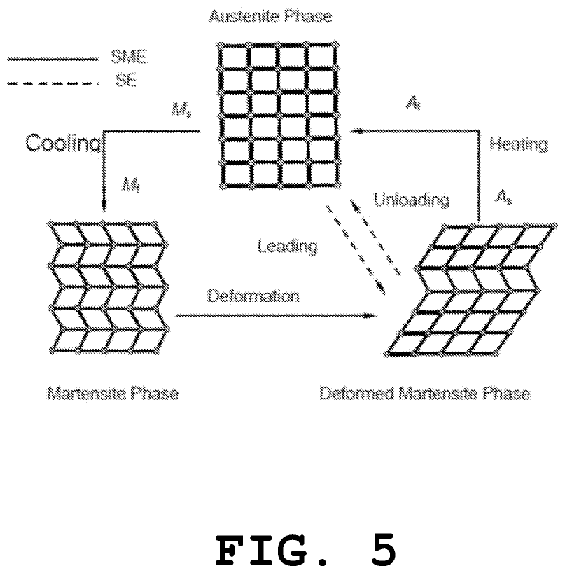

Nitinol exhibits two unique properties: shape memory effect (SME) and superelasticity (SE). These properties are governed by martensitic transformation, the solid-solid diffusionless phase transformation, see FIG. 5, where the more-ordered parent phase austenite is transformed to the less-ordered martensite phase. The phase transformation from austenite to martensite (and vice versa) is marked by four transition temperatures: the Martensite finish (M_{f}) temperature, the Martensite start (Mₛ) temperature, the Austenite finish (A_{f}) temperature, and the Austenite start (As) temperature, where M_{f}<Mₛ<Aₛ<A_{f}. A change in the temperature T within the range Mₛ<T<Aₛ results in no phase change. Both martensite and austenite may coexist at the temperature T between M_{f}<T<A_{f}.

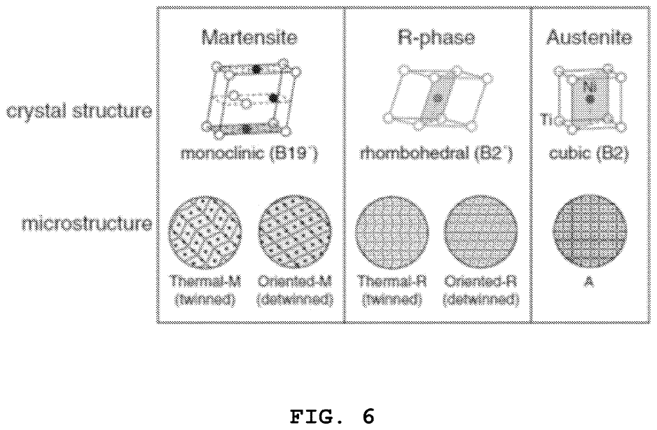

At different temperatures, different phase structures are preferred. When the Nitinol element is cooled through the transition temperature the martensite structure forms from the parent austenite structure. Austenite is the high temperature, stress-free phase characterized by an ordered B2 crystal structure (See FIG. 6). Martensite, the low temperature phase is heavily twinned and has a monoclinic B19 orthorhombic structure. Martensite can be strained up to 8% via detwinning. In the martensite phase, the Nitinol element can be strained to change its shape. That shape will be maintained until the temperature of the element is raised above the Aₛ temperature at which point, the element will convert back to Austenite and recover strain to the original shape. At T=A_{f} all the martensite has been converted back to Austenite and all the strain has been recovered.

When Nitinol is used for its superelastic properties, T>A_{f}, and thus the Nitinol is fully austenitic. When the Nitinol device is strained, the stress causes the austenite to transform to martensite (referred to as stress induced martensite). With T>A_{f}, the martensite is unstable and when the load is removed, the reverse phase transformation (martensite to austenite) occurs.

The M_{f}, Mₛ, Aₛ, and A_{f} temperature of Nitinol can be tuned by heat treating the Nitinol. Heat treating causes a Nickel-rich precipitation reaction that changes the exact chemical matrix composition.

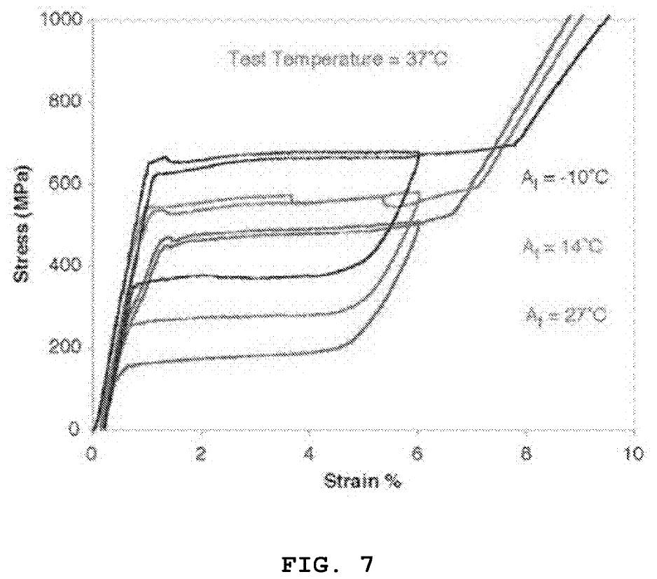

The temperature difference between the body the Nitinol device will be used in (assumed to be 37° C.) and the Nitinol's A_{f} temperature affects the mechanical hysteresis properties of the Nitinol device. A smaller temperature difference between the device's A_{f} and the body temperature will result in smaller stress levels than a larger temperature difference (See FIG. 7).

Clinically, it is often advantageous to deform the Nitinol device and then mechanically constrain the deformed shape. This allows the physician to release the mechanical constrain and as long as T>A_{f}, the device will regain its original shape.

In the case of the novel guidewire of the present invention, the tines of the guidewire are shape set via a heat treatment to create the outward flare of the tines. The austenite start temperature of the shape set region is above body temperature, thus the wire will exhibit superelastic properties when used within the body. Shape setting tines 220 may result in an austenite start temperature that is slightly greater than the rest of the Nitinol guidewire, but should still be fully austenitic at body temperature.

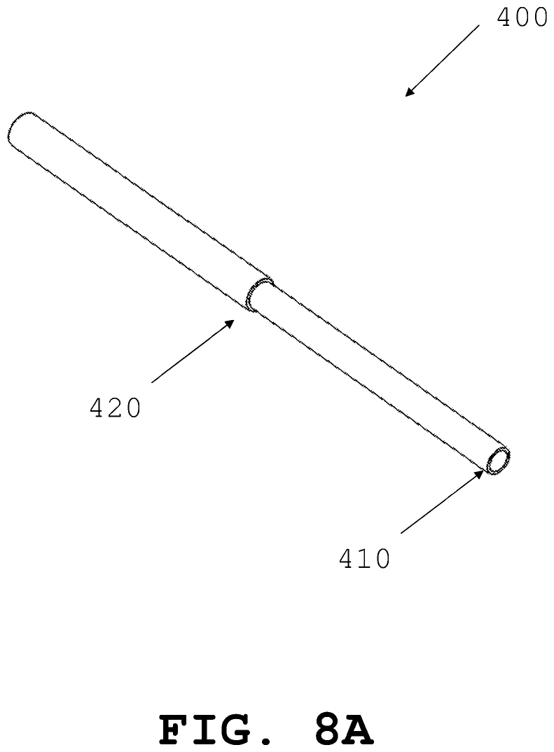

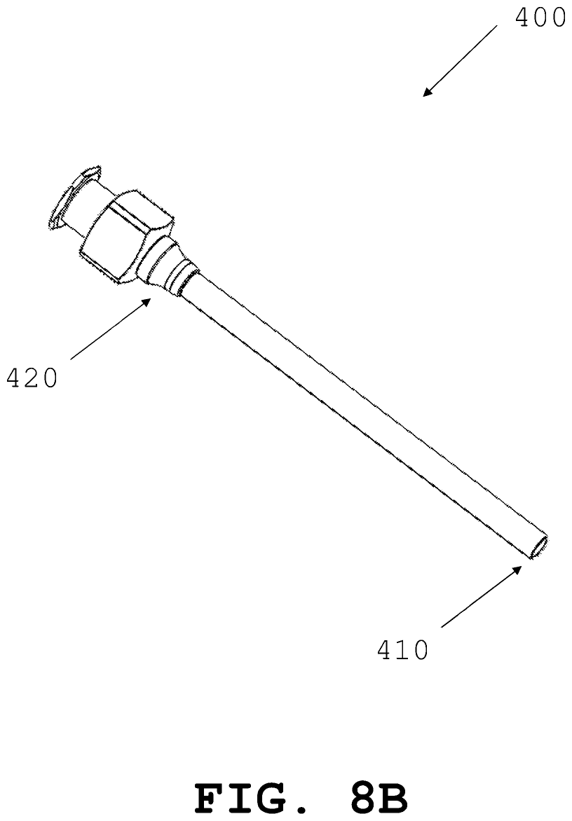

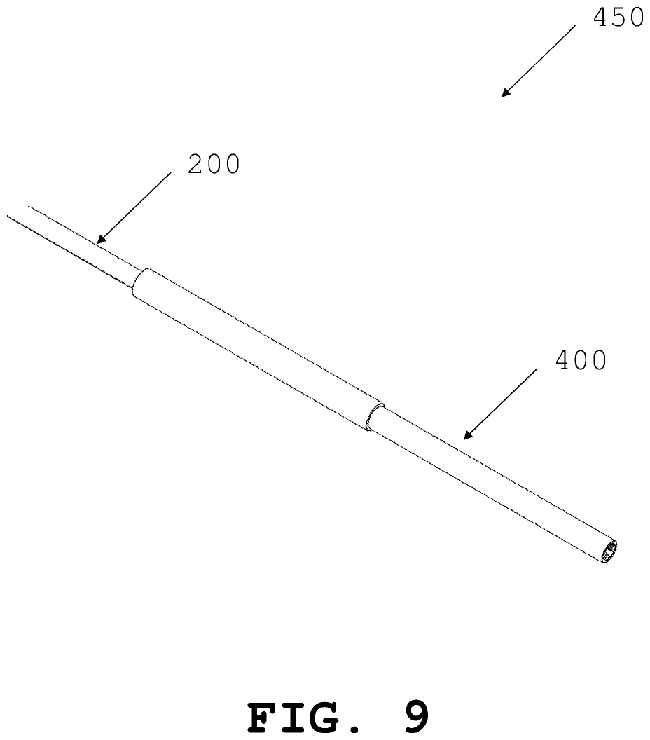

Looking now at FIGS. 8A and 8B, sheaths, 400 for constraining the guidewire tines are shown. Sheaths 400 are sized so that inner cannulation 410 is large enough to allow the guidewire 200 to fit within the central lumen of sheath 400. Furthermore, sheath 400 has a step 420 that is of a larger diameter than the rest of the sheath and as will be shown abuts the opening of a jamshidi or other similar needle to prevent the sheath from sliding into the needle.

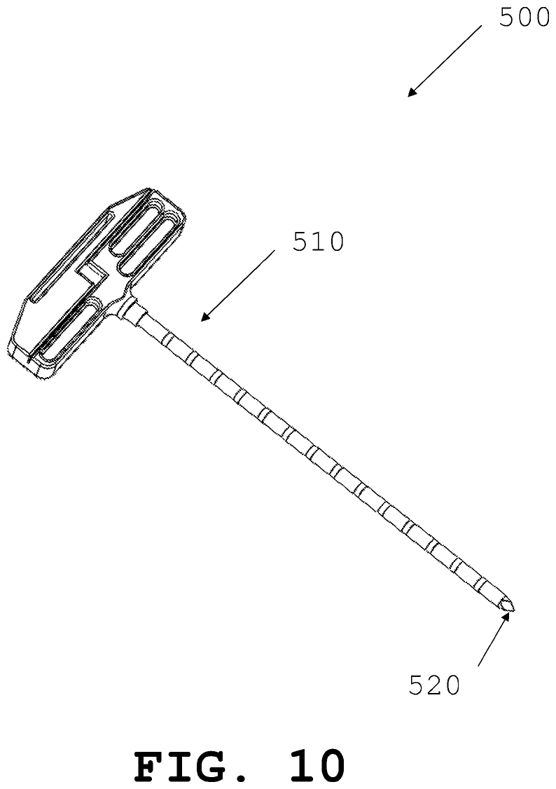

FIG. 9 shows the guidewire 200 collapsed and within the sheath 300. When the guidewire is formed from superelastic nitinol, the tines are constrained in the sheath and stored in a state of stress induced martensite. FIG. 10 shows a standard jamshidi needle 500. The jamshidi needle can be used to gain access to a site within the body. The jamshidi needle is made up of an inner stylus 520, and an outer sheath 510. Inner stylus 520 may be removed once the jamshidi needle is located in its desired location.

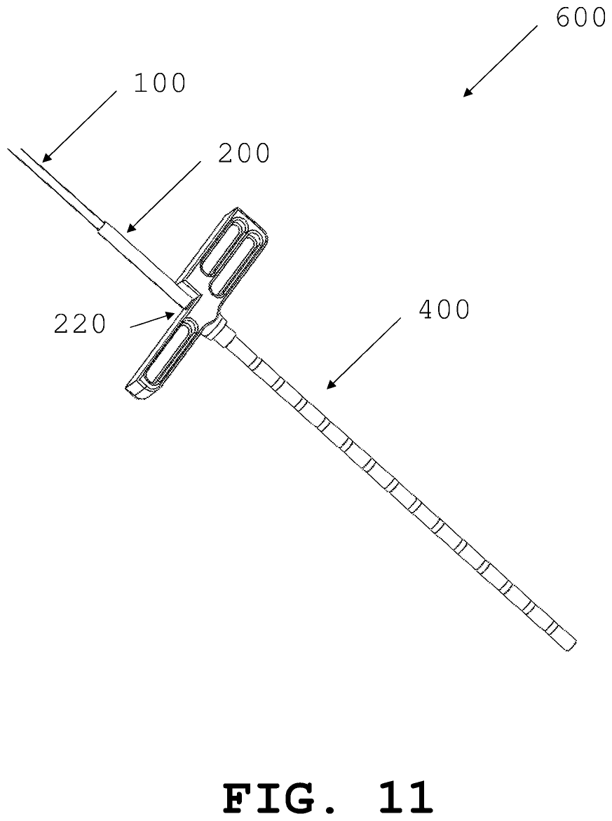

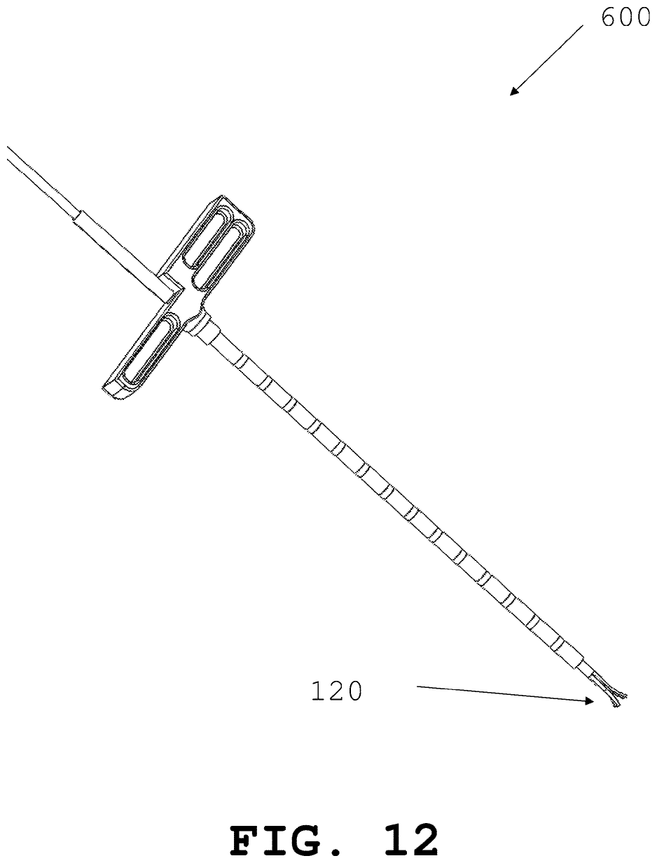

Looking now at FIG. 11, the guidewire 100 is strained within sheath 400. The inner stylus 520 of the jamshidi needle has been removed, and the strained guidewire has been introduced into the jamshidi needle up until the point that the step 420 in the sheath abuts the jamshidi needle. At this point, the guidewire 200 can be advanced, and as shown in FIG. 12 the tines 220 of the guidewire spring back open to their unconstrained state. When the guidewire is formed from nitinol, the tines return to their austenitic phase. At this point the jamshidi can be removed leaving only the guidewire in place. Surgical instruments such as drills and screw drivers can be placed over the guidewire. Implants such as screws can be implanted over the guidewire. The guidewire resists forward and reverse motion as well as rotation. When an advancing force is applied to the guidewire, the tines 220 want to splay further open and the wire resists forward motion.

When formed from Nitinol, by adjusting the heat treatment properties used to shape set the tines, the amount of force the tines exert when they recover their flared outward shape can be changed. As the A_{f} temperature of the Nitinol tines approaches the body temperature, the amount of force reduces. The temperature differential between A_{f} and body should be at least about 5° C. so that the tines have enough strength to flare open against the bone as the Nitinol recovers the strain at the lower plateau stress. In a preferred embodiment, the temperature differential between A_{f} and body is at least about 15° C.

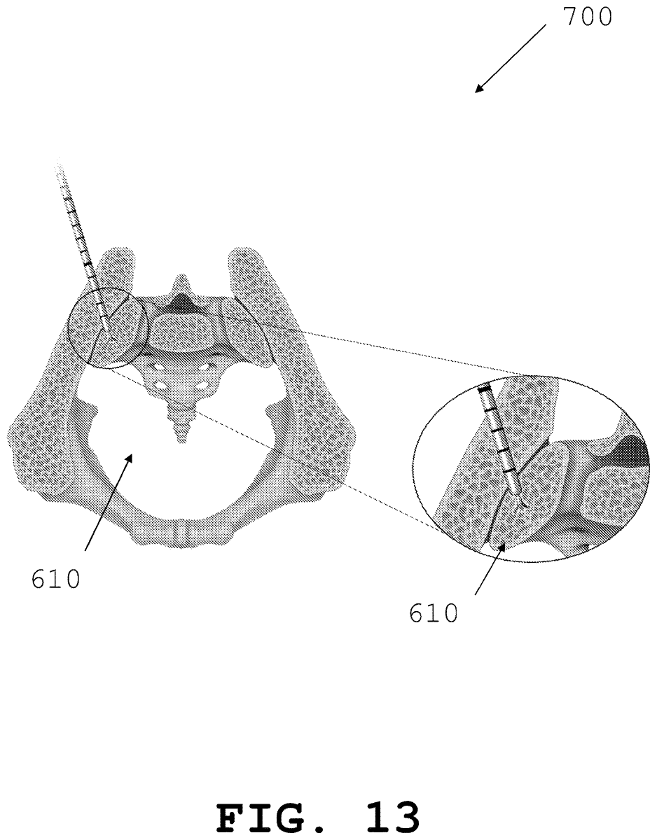

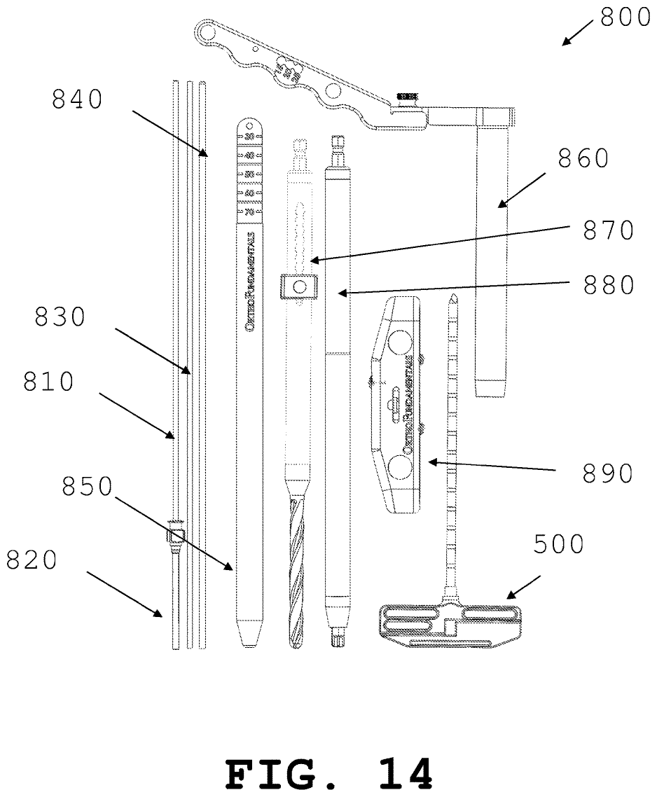

FIG. 13 shows the Nitinol guidewire of the present invention used to guide the placement of instruments and implants used in an sacroiliac joint fusion. As one can see, if the guidewire was to undesirably advance it would risk perforating the distal cortex of the sacrum 610 and entering the abdomen 620.

In accordance with one or more embodiments, a kit is disclosed. The kit may generally include an implant such as a screw. Said screw may be provided sterile, packaged in a Tyvck or other conventional sterile barrier material. Other components for clinical use as described herein may also be included in the kit. The screw and/or kit may include a washer.

In accordance with one or more embodiments, the implant may be one as described in International (PCT) Patent Application Publication No. WO2023/137124 or International (PCT) Patent Application Publication No. WO2024/006536, both to Applicant, the entire content of each of which is hereby incorporated herein by reference in its entirety for all purposes.

In accordance with one or more embodiments, various components of the kit may be configured for compatibility with the disclosed guidewire. For example, a drill bit, implant e.g. screw, dilator/sizer and/or screwdriver may be cannulated so as to be removably received by the guidewire. Specifically, one or more cannulated components can be removably received over and along the guidewire.

Kits may be constructed specifically for the intended surgical use of the screw. In one example, looking at FIG. 14, the kit 800, may be constructed for use when performing sacroiliac joint fusion using a posterolateral or oblique approach. The kit, 800, is designed to include a sterile instrument kit which may provide for example one or more of a guidewire formed in accordance with the present invention 810 and pre-constrained in sheath 820, a blunt guidewire 830, an exchange pin, 840, a combination one step tissue dilator and screw sizer, 850, a tissue shield or drill guide, 860, one or more drill bits, 870, a screwdriver, 880, and a drive handle or ratchet, 890. Additionally, the kit may include a Jamshidi or biopsy needle, 500, sized to accept guidewire 810 and sheath 820.

The contents of kit 800 may be re-usable, re-processible, or fully disposable. The contents of instrument kit 800 may be mounted on a card or thermoformed tray. The instruments and card/tray may be sealed in a sterile barrier. In a preferred embodiment, the instrument kits are sealed under vacuum so that they do not move during distribution.

The kits disclosed in FIG. 14 may further include a separately packed source of a bone growth agent or other biologically active agent. The biologically active agent may include bone growth promoting material. In some embodiments, the biologically active agent may include therapeutic agents and/or pharmacological agents for release, including sustained release, into a surrounding tissue to treat, for example, pain, inflammation and degeneration. The agents may include pharmacological agents, such as, for example, antibiotics, pain medications, analgesics, anesthetics, anti-inflammatory drugs including but not limited to steroids, anti-viral and anti-retroviral compounds, therapeutic proteins or peptides, therapeutic nucleic acids (as naked plasmid or a component of an integrating or non-integrating gene therapy vector system), and combinations thereof. In some embodiments, the agent may include bone cement that enhances fixation of the screw with tissue. In some embodiments, the bone cement may include a poly(methyl methacrylate) (PMMA); methyl methacrylate (MMA); calcium phosphate; a resorbable polymer, such as, for example, PLA, PGA or combinations thereof; a resorbable polymer with allograft, such as, for example, particles or fibers of mineralized bone and/or combinations thereof. In other applications the biologically active agent may include demineralized bone or bioactive glass.

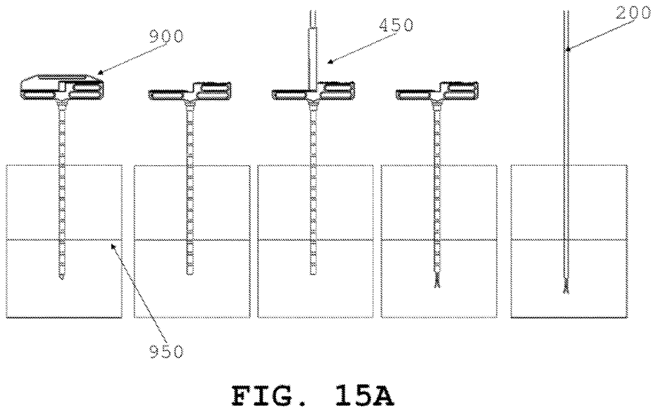

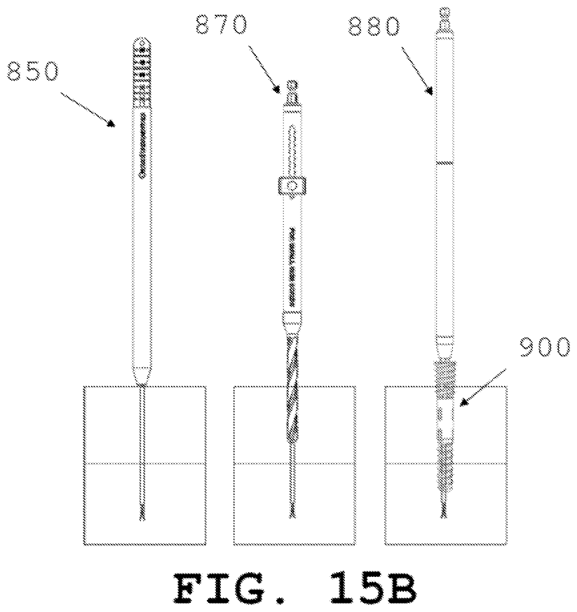

Kit 800 may be used to perform a posterolateral or oblique sacral iliac joint fusion and may include instructions for use. Kit 800 is provided sterile. Screws are also provided sterile and packaged individually and separately. In a first step (see FIG. 15A), the surgeon positions the jamshidi needle 500 across the joint 950 in its desired location to provide a through hole or cannulation. The inner stylet of the jamshidi needle is removed, and the constrained guidewire 450 is placed down the jamshidi needle. When the sheath abuts the jamshidi needle, the guidewire can be further advanced so that it exits the jamshidi needle and the tines flare open to their original unconstrained shape. The guidewire will now resist forward and reverse motion as well as rotation. The jamshidi needle can now be removed. Looking at FIG. 15B, the surgeon can slide a combination one step tissue dilator and screw sizer 850 over the guidewire until the tip abuts the ilium surface and read the length of the screw to be used. Since guidewire 200 is calibrated with a known length, the amount of the guidewire that is exposed on the backend of the dilator and screw sizer tells the physician the screw depth of the hole to drill (if drilling a hole) and the length of the screw to use. With the screw length known, the one step tissue dilator and screw sizer can be removed. A drill bit 870 can be slid over the guidewire and drilled to the appropriate length. The drill bit 870 can be removed and the screw 900 and screwdriver 880 can be slid over the guidewire. Upon implantation of the screw, the screwdriver can be removed and then the guidewire can be removed. At the end of the case the contents of the instrument kit may be discarded. In an alternative embodiment the contents of the instrument kit can be placed in a biohazard container, and the instruments can be cleaned, re-processed to like new condition, and reused.

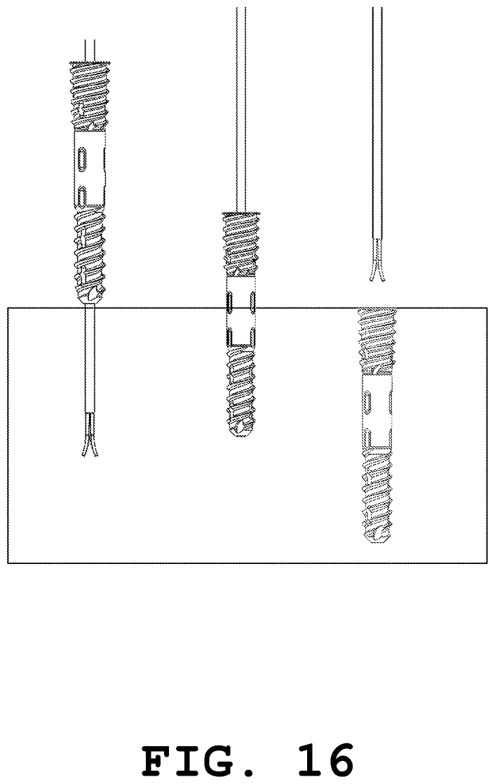

Now looking at FIG. 16, it should be appreciated that when the guidewire of the present invention has been deployed within the bone and the tines have flared open, the screw can be placed over the guidewire and the surgeon can begin screwing the screw into the bone. As the screw is advanced, the cannulation on the screw causes the tines of the guidewire to collapse within the cannulation of the screw. The screw can be fully implanted, and then the guidewire can be removed.

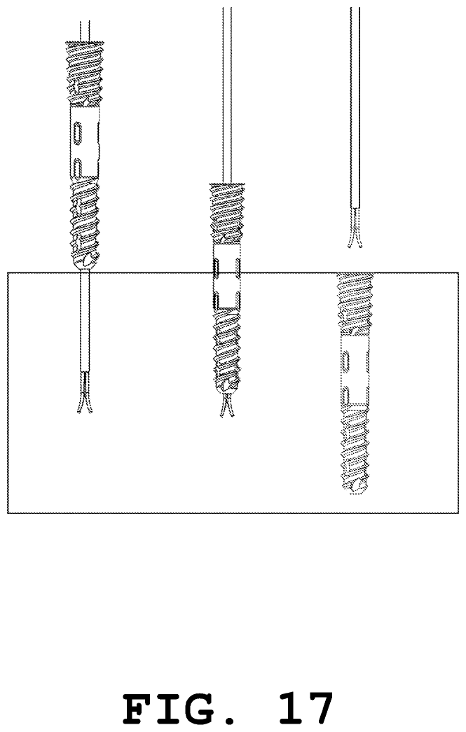

In some cases it may be undesirable to use the screw to mechanically compress the tines of the guidewire. In this case the guidewire should be removed prior to final screw placement. Now looking at FIG. 17, if the guidewire of the present invention has been deployed within the bone and the tines have flared open, the screw can be placed over the guidewire and the surgeon can begin screwing the screw into the bone. On fluoroscopy the surgeon can see when the tip of the screw is approaching the region of the guidewire that is flaring outward. When the screw is near this region, the guidewire can be removed, and the screw implantation finished. This may be a preferable method of using the novel guidewire as bone may be within the flared region of the tines making it difficult to collapse the tines and it would be possible for one or more tines to rub against the screw implant, risking breaking a tine.

It should be understood that many additional changes in the details, materials, steps and arrangements of parts, which have been herein described and illustrated in order to explain the nature of the present invention, may be made by those skilled in the art while still remaining within the principles and scope of the invention.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

What is claimed is:

1. A method of performing orthopedic surgery using a guidewire, the method comprising the steps of:
forming a cannulation or through hole in one or more targeted bones at a surgical site;
positioning a guidewire down into the cannulation or through hole, the guidewire having:
a distal tip, a proximal portion, and an expansion segment along a length of the guidewire;
the expansion segment defining a plurality of flanges being deformable from a non-expanded form to an expanded form upon advancement of the guidewire into the surgical site to resist forward and/or rotational motion of the guidewire, the non-expanded form of the expansion segment allowing for a diameter or cross-sectional area thereof to be equal to or less than a diameter or cross-section of the proximal portion of the guidewire when contained within a surgical tool or sheath, and the expanded form of the expansion segment allowing for expansion of the flanges in an outward direction to increase the cross-sectional area of the expansion segment relative to the proximal portion to resist forward advancement of the guidewire into the surgical site,
wherein any additional advancement of the guidewire promotes further deformation of the flanges in the outward direction causing further resistance to forward and/or rotational motion;
delivering an implant to the target bone(s) using the guidewire; and
removing the guidewire.

2. The method of claim 1, further comprising using an introduction sheath to facilitate positioning of the guidewire at the surgical site.

3. The method of claim 1, wherein a jamshidi needle is used to form the cannulation or through hole.

4. The method of claim 1, wherein the guidewire is removed after complete placement of the implant.

5. The method of claim 1, wherein the guidewire is removed prior to complete placement of the implant.

6. The method of claim 1, wherein the expansion segment is located proximate to the distal tip of the guidewire such that the expansion segment is positioned between the proximal portion and the distal tip of the guidewire, wherein the proximal portion and the distal tip of the guidewire are both fully formed and do not define flanges.

7. The method of claim 1, wherein the expansion segment is located at the distal tip of the guidewire such that the distal tip defines the plurality of flanges.

8. The method of claim 1, wherein the implant is an orthopedic screw.

9. The method according to claim 1, wherein the targeted bone(s) at the surgical site are associated with a sacroiliac (SI) joint fusion procedure.

10. The method of claim 1, wherein the guidewire is made of Nitinol.

11. The method of claim 1, wherein the guidewire has a non-circular cross-section to provide additional resistance to rotation at the surgical site.

12. The method of claim 1, wherein the guidewire is capable of resisting advancement when loaded in excess of 10 lbs.

13. The method of claim 1, further comprising drilling the one or more targeted bone(s) with a drill positioned over the guidewire to define a hole for implantation.

14. A guidewire for facilitating orthopedic surgical procedures, the guidewire comprising:
a distal tip, a proximal portion, and an expansion segment along a length of the guidewire;
the expansion segment defining a plurality of flanges being deformable from a non-expanded form to an expanded form upon advancement of the guidewire into a surgical site to resist forward and/or rotational motion of the guidewire, the non-expanded form of the expansion segment allowing for a diameter or cross-sectional area thereof to be equal to or less than a diameter or cross-section of the proximal portion of the guidewire when contained within a surgical tool or sheath, and the expanded form of the expansion segment allowing for expansion of the flanges in an outward direction to increase the cross-sectional area of the expansion segment relative to the proximal portion to resist forward advancement of the guidewire into the surgical site,
wherein any additional advancement of the guidewire promotes further deformation of the flanges in the outward direction causing further resistance to forward and/or rotational motion.

15. The guidewire of claim 14, wherein the expansion segment is located proximate to the distal tip of the guidewire such that the expansion segment is positioned between the proximal portion and the distal tip of the guidewire, wherein the proximal portion and the distal tip of the guidewire are both fully formed and do not define flanges.

16. The guidewire of claim 14, wherein the expansion segment is located at the distal tip of the guidewire such that the distal tip defines the plurality of flanges.

17. The guidewire of claim 14, wherein the expansion segment is cut and shape set to define three or more tines, said tines constructed and arranged to flare away from a central axis of the guidewire.

18. The guidewire of claim 14, constructed and arranged to resist forward and/or rotational motion when in place at a surgical site.

19. The guidewire of claim 14, further comprising a sheath for constraining the expansion segment.

20. The guidewire of claim 19, wherein the sheath is sized to fit at least partially within a jamshidi needle, wherein advancing the guidewire out the end of the sheath that is partially within a jamshidi needle allows the guidewire tines to return to their unconstrained shape.

21. The guidewire according to claim 14, constructed and arranged to facilitate placement of instruments and/or implants during an orthopedic surgical procedure.

22. The guidewire according to claim 21, wherein the orthopedic procedure is a sacroiliac (SI) joint fusion procedure.

23. The guidewire of claim 14, wherein the guidewire is made of Nitinol.

24. The guidewire of claim 14, having a non-circular cross-section to provide additional resistance to rotation at a surgical site.

25. An orthopedic surgical kit comprising the guidewire of any of the preceding claims.

26. The kit of claim 25, further comprising an orthopedic implant.

27. A method of facilitating orthopedic surgery, comprising providing the guidewire of any of the preceding claims.
